(19) United States
(12) Patent Application Publication
(10) Pub. No.: US 2024/0052293 A1
(43) Pub. Date: Feb. 15, 2024

(54) BIOLOGICAL REACTION SYSTEMS

(71) Applicant: ALIT BIOTECH (SHANGHAI) CO., LTD., Shanghai (CN)

(72) Inventors: Yu LIU, Shanghai (CN); Rui CHEN, Shanghai (CN)

(73) Assignee: ALIT BIOTECH (SHANGHAI) CO., LTD., Shanghai (CN)

(21) Appl. No.: 18/448,133

(22) Filed: Aug. 10, 2023

(30) Foreign Application Priority Data
Aug. 11, 2022 (CN) 202210963148.X
Aug. 11, 2022 (CN) 202222117290.0
Aug. 11, 2022 (CN) 202222129463.0

(51) Int. Cl. C12M 1/00; C12M 1/02; C12M 1/34; C12M 1/06; C12M 1/107

(52) U.S. Cl. CPC C12M 47/18; C12M 41/22; C12M 41/26; C12M 27/02; C12M 23/40; C12M 21/04

(57) ABSTRACT

The present disclosure provides a biological reaction system. The biological reaction system may include a reaction vessel and a temperature sensor. The reaction vessel may include an accommodating chamber configured to accommodate a mixture of cells and liquids. An outer side of a bottom of the reaction vessel may be provided with a groove recessed in a direction towards the accommodating chamber. The temperature sensor may include a probe, and the probe may be within the groove. The groove may be provided with a first heat conduction member, and the first heat conduction member may be sleeved outside the probe.

### CROSS-REFERENCE TO RELATED APPLICATIONS

This application claims priority to the Chinese Patent Application No. 202210963148.X, filed on Aug. 11, 2022, Chinese Patent Application No. 202222129463.0, filed on Aug. 11, 2022, and Chinese Patent Application No. 202222117290.0, filed on Aug. 11, 2022, the contents of each of which are hereby incorporated by reference.

### TECHNICAL FIELD

The present disclosure relates to a technology field of biological reactions, in particular, relates to biological reaction systems.

### BACKGROUND

A biological reaction system is a device system for expanding cells, and monitoring and maintaining a cellular physiological status. After a genetic modification or genetic editing, a cellular tissue may be placed in the biological reaction system for cell expansion. During a process of cell expansion, an appropriate temperature needs to be controlled. However, due to limitation of an inherent morphology and structure of the biological reaction system, it is usually difficult to obtain an accurate temperature and precisely regulate the temperature.

Therefore, it is desired to provide biological reaction systems to achieve temperature measurements and temperature regulations accurately.

### SUMMARY

According to an aspect of the present disclosure, a biological reaction system may be provided. The biological reaction system may include a reaction vessel and a temperature sensor. The reaction vessel may include an accommodating chamber configured to accommodate a mixture of cells and liquids. An outer side of a bottom of the reaction vessel may be provided with a groove recessed in a direction towards the accommodating chamber. The temperature sensor may include a probe, and the probe may be within the groove. The groove may be provided with a first heat conduction member, and the first heat conduction member may be sleeved outside the probe.

In some embodiments, the biological reaction system may further include a second heat conduction member. At least part of the second heat conduction member may be located between the probe and the first heat conduction member. A portion of an inner surface of the second heat conduction member may be adhered with the probe, and a portion of an outer surface of the second hear conduction member may be adhered with an inner surface of the first heat conduction member.

In some embodiments, the groove may be further provided with a flexible sealing member, the sealing member may be abutted against the first heat conduction member, and the sealing member may be in an interference fit with the groove

In some embodiments, a ratio of a depth of the groove to a diameter of the bottom of the reaction vessel may be within a range from 1:30-1:2.5.

In some embodiments, the biological reaction system may include an elastic compaction mechanism. The elastic compaction mechanism may include a fixing portion, a compaction-abutting portion, and an elastic member. A position of the fixing portion may be fixed, the elastic member may be abutted between the fixing portion and the compaction-abutting portion, and the compaction-abutting portion may be configured to abut and press the first conduction member under an elastic force of the elastic member.

In some embodiments, an inner wall of the first conduction member may be adapted to a shape of the probe.

In some embodiments, the inner wall of the first conduction may be provided with an insulating layer, and the insulating layer may be adhered with the probe.

In some embodiments, the biological reaction system may further include a heating mechanism and a controller. The heating mechanism may be adhered with at least part of an outer wall of the reaction vessel, the heating mechanism and the temperature sensor may be connected with the controller, and the controller may control a heating temperature of the heating mechanism based on a temperature sensed by the temperature sensor.

In some embodiments, the biological reaction system may further include a dissolved oxygen electrode and a first PH electrode. The dissolved oxygen electrode and the first PH electrode may be arranged within the accommodating chamber of the reaction vessel and located on a sidewall of the reaction vessel.

In some embodiments, the biological reaction system may further include a second PH electrode. The reaction vessel may include an upper cover configured to seal the accommodating chamber, the upper cover may include a detecting port, the second PH electrode may be inserted into the accommodating chamber through the detecting port, and a sealing device may be arranged between the second PH electrode and the upper cover of the reaction vessel.

In some embodiments, the biological reaction system may further include a driving component and an agitating device. The agitating device may include a central rotating shaft and a plurality of agitating paddle assemblies. For each of the plurality of agitating paddle assemblies, the agitating paddle assembly may include an installation portion and a paddle portion arranged on the installation portion, the installation portion may be provided with a first fixing member, the central rotating shaft may be provided with a second fixing member, and the first fixing member and the second fixing member may be connected through a snap-fit.

In some embodiments, the biological reaction system may further include a plurality of gas filters. The plurality of gas filters may be in communication with the accommodating chamber, and positions of the plurality of gas filters may be relatively fixed to each other.

In some embodiments, the biological reaction system may further include a tail gas processing device.

### BRIEF DESCRIPTION OF THE DRAWINGS

This specification will be further illustrated by way of exemplary embodiments, which will be described in detail with the accompanying drawings. These examples are non-limiting, and in these examples, the same number indicates the same structure, wherein:

FIG. 1 is a schematic diagram illustrating an exemplary structure of a biological reaction system according to some embodiments of the present disclosure;

FIG. 2 is a schematic diagram illustrating an exemplary structure of a part of a biological reaction system according to some embodiments of the present disclosure;

FIG. 3 is a cross-sectional view illustrating an exemplary structure of a part of a biological reaction system according to some embodiments of the present disclosure;

FIG. 4 is a schematic diagram illustrating an exemplary structure of an elastic compaction mechanism according to some embodiments of the present disclosure;

FIG. 5 is a schematic diagram illustrating an exemplary dissolved oxygen electrode, an exemplary first PH electrode, and an exemplary second PH electrode according to some embodiments of the present disclosure;

FIG. 6 is a schematic diagram illustrating an exemplary structure of an agitating device according to some embodiments of the present disclosure;

FIG. 7 is a schematic splicing diagram illustrating an exemplary agitating paddle assembly according to some embodiments of the present disclosure;

FIG. 8 is a schematic diagram illustrating an installation of an exemplary agitating paddle assembly according to some embodiments of the present disclosure;

FIG. 9 is a schematic diagram illustrating another exemplary structure of an agitating device according to some embodiments of the present disclosure;

FIG. 10 is a schematic diagram illustrating an exemplary structure of a plurality of gas filters according to some embodiments of the present disclosure; and

FIG. 11 is a schematic diagram illustrating an exemplary structure of a plurality of tracheas and a trachea coupling member according to some embodiments of the present disclosure.

### DETAILED DESCRIPTION

In order to more clearly illustrate the technical solutions of the embodiments of the present specification, the following briefly introduces the drawings that need to be used in the description of the embodiments. Apparently, the accompanying drawings in the following description are only some examples or embodiments of this specification, and those skilled in the art can also apply this specification to other similar scenarios. Unless obviously obtained from the context or the context illustrates otherwise, the same numeral in the drawings refers to the same structure or operation.

It should be understood that “system”, “device”, “unit” and/or “module” as used herein is a method for distinguishing different components, elements, parts, parts or assemblies of different levels. However, the words may be replaced by other expressions if other words can achieve the same purpose.

As indicated in the specification and claims, the terms “a”, “an”, “an” and/or “the” are not specific to the singular and may include the plural unless the context clearly indicates an exception. Generally speaking, the terms “comprising” and “comprising” only suggest the inclusion of clearly identified steps and elements, and these steps and elements do not constitute an exclusive list, and the method or device may also contain other steps or elements.

A flowchart is used in the present disclosure to illustrate the operations performed by the system according to the embodiments of the present disclosure. It should be understood that the preceding or following operations may not be performed accurately in sequence. On the contrary, each step can be processed in reverse order or simultaneously. At the same time, other operations can also be added to these processes, or a step or several operations can be removed from these processes.

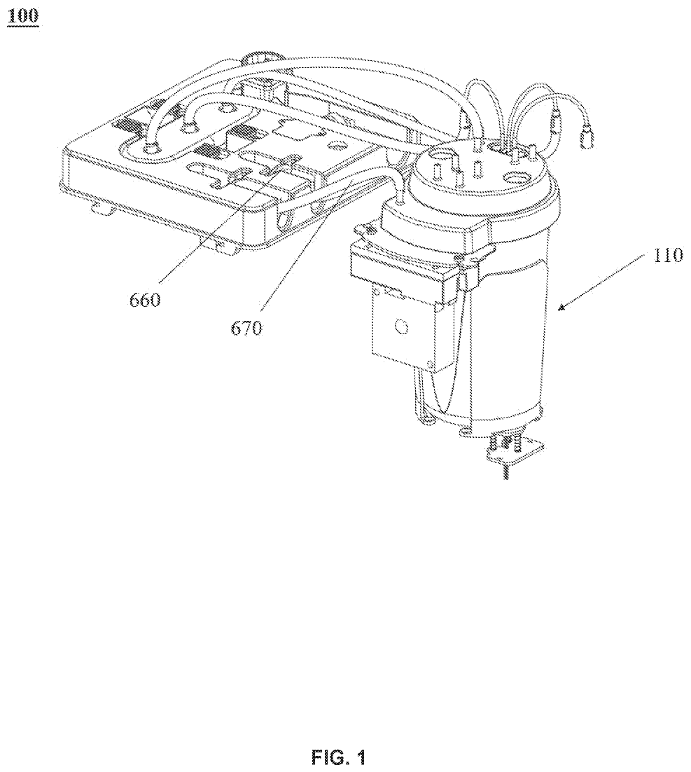

FIG. 1 is a schematic diagram illustrating an exemplary structure of a biological reaction system 100 according to some embodiments of the present disclosure. The biological reaction system 100 may be configured to expand cells, and monitor and maintain a cellular physiological status. As shown in FIG. 1, the biological reaction system 100 may include a reaction vessel 110, an agitating device arranged in the reaction vessel 110, an assembly configured to control an inner temperature of the reaction vessel 110, and a pipeline and a filter assembly configured to feed or deliver gas or materials.

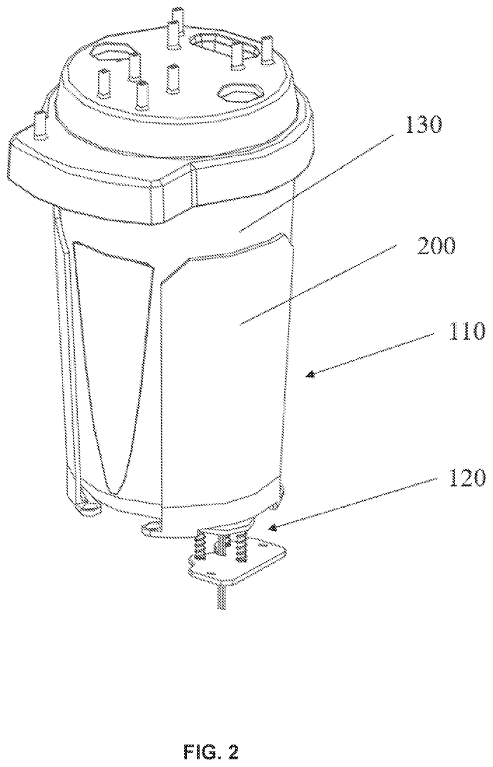

FIG. 2 is a schematic diagram illustrating an exemplary structure of a part of a biological reaction system 100 according to some embodiments of the present disclosure.

As shown in FIG. 2, in some embodiments, the biological reaction system 100 may include a reaction vessel 110 and a temperature sensor 120.

The reaction vessel 110 may be a vessel configured to accommodate reactants. The reactants may include a mixture of cells to be expanded and liquids (e.g., a nutrient solution required for cell growth, etc.). In some embodiments, the reaction vessel 110 may include an accommodating chamber 130 configured to accommodate the mixture of cells and liquids.

The temperature sensor 120 refers to a sensor that is configured to detect a temperature and convert the temperature into a usable output signal. In some embodiments, the temperature sensor 120 may be configured to measure the temperature in the reaction vessel 100 to facilitate an operator to understand the temperature in the reaction vessel 100 in real-time and further to facilitate a real-time regulation of the temperature in the reaction vessel 110, so that a suitable cell growth temperature may be kept in the reaction vessel 100, which is conducive to the cell expansion. In some embodiments, the temperature sensor 120 may include a probe 140. The probe 140 may be an assembly configured to contact the reaction vessel 110 directly or indirectly to detect the temperature.

In some embodiments, as shown in FIG. 2, an outer side of a bottom wall of the reaction vessel 110 may be provided with a groove recessed in a direction towards the accommodating chamber 130, and the probe 140 may be arranged in the groove. The probe 140 may detect the temperature in the reaction vessel 110 indirectly to avoid a direct contact between the probe 140 and the mixture in the reaction vessel 110. The direct contact affects the mixture in the reaction vessel 110.

In some embodiments, an outer side of a sidewall of the reaction vessel 110 may be provided with a sidewall groove recessed in the direction towards the accommodating chamber 130, and the probe 140 may be arranged in the sidewall groove.

In some embodiments, a position of the groove may be arranged as close as possible to a position of the mixture in the reaction vessel 110. Furthermore, the position of the groove may be arranged as close as possible to a center of the mixture without affecting functions of other structures in the accommodating chamber 130.

In some embodiments, a part of the groove toward the inside of the accommodating chamber 130 may be in a shape of a circular arc, and an arc-shaped connecting position may be arranged between the groove and a wall of the accommodating chamber 130 to prevent cells from adhering to edges, corners, and gaps, which is conducive to the cell expansion.

In some embodiments, a wall of the groove may be made of the same material as a sidewall and a bottom wall of the accommodating chamber 130. That is, the wall of the groove and the wall of the accommodating chamber 130 may be integrally formed.

In some embodiments, a material of the wall of the groove may be different from a material of the sidewall and the bottom wall of the accommodating chamber 130. A thermal conductivity of the wall of the groove may be superior to a thermal conductivity of the sidewall and the bottom wall of the accommodating chamber 130. For example, the wall of the grove may be made of a metallic material. Based on the arrangement illustrated above, the temperature detected by the probe 140 may be accurate, which may ensure an usage performance of the sidewall and the bottom wall of the accommodating chamber 130.

In some embodiments, a plurality of grooves may be provided. In some embodiments, the plurality of grooves may be distributed on the bottom wall of the reaction vessel 110, and the plurality of grooves may also be distributed on the sidewall of the reaction vessel 110. In some embodiments, at least part of the plurality of grooves may be distributed on the bottom wall of the reaction vessel 110, and the rest of the plurality of grooves may also be distributed on the sidewall of the reaction vessel 110. In some embodiments, each groove may be arranged with a probe 140 correspondingly to achieve multi-point temperature measurement for the reaction vessel 110.

It should be noted that relevant structures (e.g., a first heat conduction member 150, etc.) of the grooves on the bottom wall of the reaction vessel 110 in the present disclosure may also be applied to a sidewall groove on the sidewall of the reaction vessel 110.

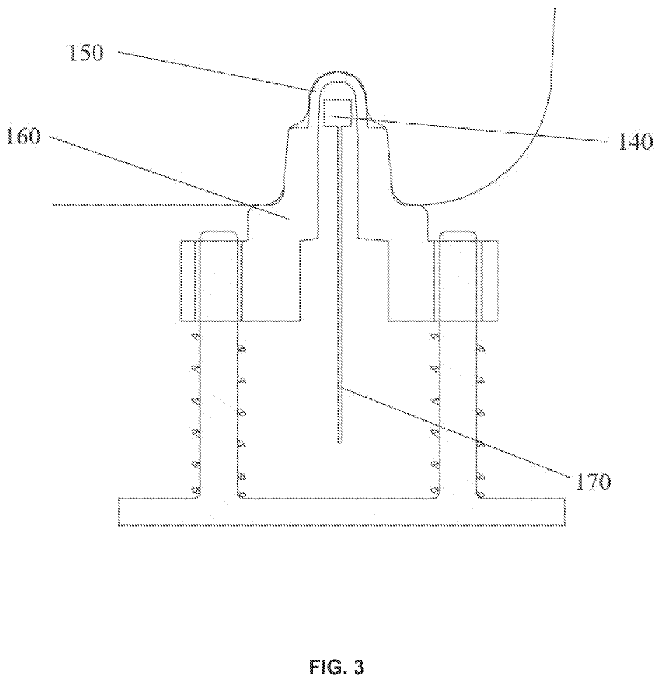

FIG. 3 is a cross-sectional view illustrating an exemplary structure of a part of a biological reaction system 100 according to some embodiments of the present disclosure. As shown in FIG. 3, in some embodiments, the groove may be provided with a first heat conduction member 150, the first heat conduction member 150 may be sleeved outside the probe 140, and the first heat conduction member 150 may perform a heat conduction between the reaction vessel 110 and the probe 140. The temperature of the mixture may be detected by conducting the temperature of the mixture through the first heat conduction member 150 to the probe 140 of the temperature sensor 120, thereby enabling the temperature sensor 120 to accurately measure the temperature and avoiding a direct contact with the mixture to affect the mixture.

In some embodiments, a shape of an inner wall of the first heat conduction member 150 may be adapted to a shape of the probe 140. In some embodiments, a shape of an outer wall of the first heat conduction member 150 may match a shape of the groove. In some embodiments, another heat conduction member may be arranged between the first heat conduction member 150 and the groove. For example, the another heat conduction member may be a thermal conductive silicone component, a thermal conductive metal component, or the like.

In some embodiments, the first heat conduction member 150 may be a hat-shaped object made of materials with a relatively good thermal conductivity (e.g., metal), an inner wall of the hat shaped object may be in complete contact with the probe 140, and an outer wall of the hat shaped object may be in complete contact with an outer wall of the groove to perform the heat conduction.

In some embodiments, due to the good thermal conductivity and the ability of the first heat conduction member 150 to adhere to the probe 140 and the groove respectively, the temperature in the reaction vessel 110 may be almost completely transmitted to the probe 140 to be detected, thereby ensuring an accurate temperature measurement by the probe 140.

In some embodiments, an inner wall of the first heat conduction member 150 may be provided with an insulating layer, and the insulating layer may be adhered with the probe 140. In some embodiments, a thermal conductivity of the insulating layer may be greater than or equal to 0.5 W/(m*k). In some embodiments, an arrangement of the insulating layer may avoid conductivity and improve safety. For example, when a failure of the probe 140 occurs and the conduction occurs, the insulating layer may prevent a direct contact between the probe 140 and the first heat conduction member 150 (e.g., a conductive metal), thereby avoiding safety hazards.

In some embodiments, a material of the insulating layer may be a material with good insulation and thermal conductivity to achieve good insulation without affecting thermal conductivity. For example, the material of the insulating layer may be a thermal conductive silicone grease, or the like.

In some embodiments, a ratio of a depth of the groove to a diameter of a bottom of the reaction vessel 110 may be within a range from 1:30-1:2.5.

In some embodiments, the ratio of the depth of the groove to the diameter of the bottom of the reaction vessel 110 may be within a range from 1:30-1:2.5. For example, the diameter of the bottom of the reaction vessel 110 may be 10 cm and the depth of the groove may be 1 cm. As another example, the diameter of the bottom of the reaction vessel 110 may be 25 cm and the depth of the groove may be 1 cm. In some embodiments, when the depth of the groove is within the depth range illustrated above, the probe 140 may fully detect the temperature in the reaction vessel 110, while the depth of the groove does not affect the mixture in the reaction vessel 110.

In some embodiments, the biological reaction system 100 may further include a second heat conduction member. The second heat conduction member may be configured to fill a space between the first heat conduction member 1500 and the probe 140 to enhance thermal conductivity. In some embodiments, at least part of the second heat conduction member may be located between the probe 140 and the first heat conduction member 150, a portion of an inner surface of the second heat conduction member may be adhered with the probe 140, and a portion of an outer surface of the second heat conduction member may be adhered with an inner surface of the first heat conduction member 150. In some embodiments, a material of the second heat conduction member may be a material with good thermal conductivity and certain fluidity, which is conducive to filling gaps. For example, the material of the second heat conduction member may be thermal conductive silicone grease, or the like. In some embodiments, a thermal conductivity of the second heat conduction member may be greater than or equal to 0.5 W/(m*k).

In some embodiments, the arrangement of the second heat conduction member may make a gap between the probe 140 and the first heat conduction member 150 be fully filled, thereby enabling good temperature conduction.

In some embodiments, the groove may be further provided with a flexible sealing member 160, the first heat conduction member 150 may be abutted against the groove through the sealing member 160, so that a gap between the groove and the first heat conduction member 150 may be sealed. In some embodiments, a shape of the sealing member 160 may match the groove, and the sealing member 160 may be in an interference fit with the groove. Under a flexible and elastic action of the sealing member 160, the first heat conduction member 150 may be closely adhered with the groove.

In some embodiments, the flexible sealing member 160 may be configured to fix a position of the first heat conduction member 150, so that the first heat conduction member 150 may be closely adhered with the groove to enhance thermal conductivity.

In some embodiments, the temperature sensor 120 may further include a connecting rod 170. The probe 140 may be arranged on an end of the connecting rod 170, another end of the connecting rod 170 may be connected with a processing device, a display of a temperature signal, or the like, and a cable may be arranged within the connecting rod 170. In some embodiments, the connecting rod 170 may pass through the sealing member 160, and at least part of the second heat conduction member may be located between the connecting rod 170 and the sealing member 160.

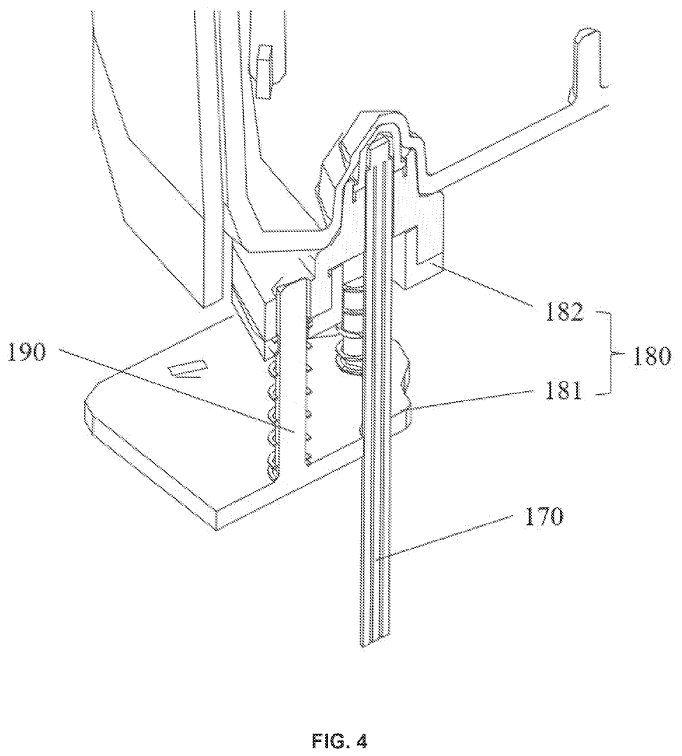

FIG. 4 is a schematic diagram illustrating an exemplary structure of an elastic compaction mechanism according to some embodiments of the present disclosure. As shown in FIG. 4, in some embodiments, the biological reaction system 100 may further include an elastic compaction mechanism 180. The elastic compaction mechanism 180 may be configured to compress the sealing member 160 to enhance the fit and tightness between the sealing member 160 and the groove.

In some embodiments, the elastic compaction mechanism 180 may include a fixing portion 181, a compaction-abutting portion 182, and an elastic member. A position of the fixing portion 181 is fixed, and the fixed position may refer to a relative fixation to an operating table or other supporting devices. In some embodiments, the fixing portion 181 may be in a shape of plate, and the plate-shaped fixing portion 181 may be installed and fixed on the table or other devices. The compaction-abutting portion 182 refers to an assembly abutted against the sealing member 160. The elastic member refers to an assembly with elastic potential energy. For example, the elastic member may be a spring, a rubber body, or the like.

In some embodiments, the elastic member may be abutted between the fixing portion 181 and the compaction-abutting portion 182, the compaction-abutting portion 182 may be configured to compress the sealing member 160 under an elastic force of the elastic member to make the sealing member 160 tightly abut against the groove. In addition, the elastic force of the elastic member may act on the sealing member 160, which may automatically tighten the sealing member 160 and reduce installation accuracy requirements of the sealing member 160.

In some embodiments, a plurality of sealing members 160 may be provided, such as two, three, five, or the like. In some embodiments, a count of the sealing members 160 may be at least three. A distance between any two elastic members among the three elastic members may be equal, so that the elastic members may be uniformly stressed, thereby improving the stability of the elastic compaction mechanism 180.

In some embodiments, the compaction-abutting portion 182 may be provided with a first through-hole, the fixing portion 181 may be provided with a second through-hole, and the connecting rod 170 may be penetrated into the groove through the first through-hole and the second through-hole.

In some embodiments, a guiding rod 190 may be arranged between the fixing portion 181 and the compaction-abutting portion 182, and the elastic member (e.g., a spring) may be sleeved on the guiding rod 190.

In some embodiments, the arrangement of the elastic compression mechanism 180 may reduce a requirement for manufacturing and assembly accuracy of the biological reaction system 100, reduce the operational difficulty of the operator, and increase the redundancy of the system. Under even more severe conditions, the sealing member 160 may still be well abutted and tightened in the groove, thereby preventing the transformation from heat conduction to heat convection in a manner of heat exchange required by the design, and reducing the accuracy and response speed of the temperature measurement. For example, a more severe condition may be that the sealing member 160 is not installed properly, the sealing member 160 loses flexibility, or the like.

In some embodiments, another alternative solution of the elastic compaction mechanism may be provided. For example, a distance adjustment structure may be arranged between the fixing portion 181 and the compaction-abutting portion 182, and the sealing member 160 may be abutted and tightened in the groove by adjusting the distance between the fixing portion 181 and the compaction-abutting portion 182. In some embodiments, the compaction-abutting portion 182 may have a certain elasticity to apply elastic compression to the sealing member 160. In some embodiments, the distance adjustment structure may include a micromotor, and the controller may adjust the distance between the fixing portion 181 and the compaction-abutting portion 182 through the micromotor, thereby adjusting a compression degree of the sealing member 160.

In some embodiments, a pressure sensor used to monitor the pressure exerted on the sealing member 160 may be provided. The controller may adjust the distance between the fixing portion 181 and the compaction-abutting portion 182 through the micromotor based on a real-time pressure obtained by the pressure sensor, thereby achieving adaptive adjustment of the compression degree of the compaction-abutting portion 182 to the sealing member 160.

In some embodiments, the biological reaction system 100 may also include a heating mechanism 200 and a controller, as shown in FIG. 2. The heating mechanism 200 may be configured to heat the biological reaction system 100 to maintain the temperature of the biological reaction system 100. The controller may be configured to control the heating mechanism 200 to be turned on or turned off. In some embodiments, the heating mechanism 200 and the temperature sensor 120 may be connected with the controller, so that the controller may control a heating temperature of the heating mechanism 200 based on the temperature detected by the temperature sensor 120.

Merely by way of example, the controller may determine whether a current temperature is within an appropriate temperature range based on the current temperature measured by the temperature sensor 120. If the current temperature is less than a lowest value of the appropriate temperature range, the heating mechanism 200 may be turned on to heat the biological reaction system 100 until the current temperature measured by the temperature sensor 120 reaches a preset temperature and then the heating may be stopped. The appropriate temperature may be preset according to the needs of cell growth, and the preset temperature may be any temperature value within the preset appropriate temperature range. For example, the preset temperature may be a highest value of the appropriate temperature range.

In some embodiments, the heating mechanism 200 may be adhered with at least part of the outer wall of the reaction vessel 110 to increase a heating range, improve heating efficiency, and ensure stable and uniform heating of the heating mechanism 200.

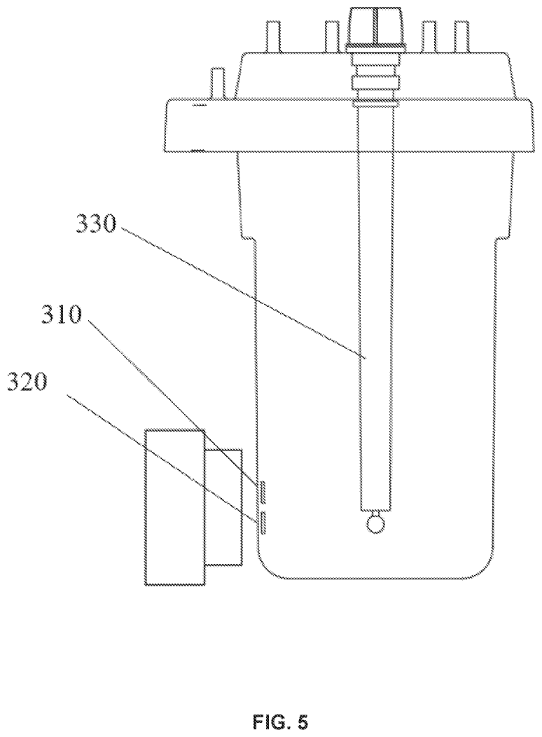

FIG. 5 is a schematic diagram illustrating an exemplary dissolved oxygen electrode 310, an exemplary first PH electrode 320, and an exemplary second PH electrode 330 according to some embodiments of the present disclosure. As shown in FIG. 5, in some embodiments, the biological reaction system 100 may further include a dissolved oxygen electrode 310 and a first PH electrode 320.

In some embodiments, the dissolved oxygen electrode 310 and the first PH electrode 320 may be arranged within the accommodating chamber 130 of the reaction vessel 110 and arranged on the sidewall of the reaction vessel 110. Merely by way of example, the dissolved oxygen electrode 310 and the first PH electrode 320 may be patch electrodes. A patch electrode may contact with the mixture to detect a dissolved oxygen content and a PH value in the mixture, which may facilitate the monitoring of the cell growth environment.

In some embodiments, the dissolved oxygen electrode 310 and the first PH electrode 320 do not contact with the mixture except for a patch portion, which may reduce the impact on the mixture.

In some embodiments, a distance between the electrode patches of the dissolved oxygen electrode 310 and the first PH electrode 320 and the bottom of the reaction vessel 110 may be set to enable the electrode patches of the dissolved oxygen electrode 310 and the first PH electrode 320 to be contacted with the mixture better, thereby monitoring parameters of the mixture.

In some embodiments, the biological reaction system 100 may further include a second PH electrode 330.

In some embodiments, the reaction vessel 110 may include an upper cover configured to close the accommodating chamber 130. The upper cover may include a detecting port, the second PH electrode 330 may be inserted into the accommodating chamber 130 through the detecting port, so that an element configured to detect PH of the second PH electrode 330 may be contacted with the mixture to detect a PH value of the mixture.

In some embodiments, the second PH electrode 330 may be fixedly installed at the detecting port on the upper cover of the reaction vessel 110. For example, the second PH electrode 330 may be fixed and installed through a snap-fit, a bolt, or the like. In some embodiments, a sealing device may be provided between the second PH electrode 330 and the upper cover of the reaction vessel 110, and the sealing device may be configured to seal the accommodating chamber 130 of the reaction vessel 110. In some embodiments, the sealing device may be a flexible sealing ring in a shape of O, which may achieve a sealing effect by abutting against the second PH electrode 330 and the upper cover respectively.

In some embodiments, the arrangement of the second PH electrode 330 with a plug-in installation manner may be further used to detect PH values at different patch positions of the first PH electrode 320 in the mixture. In addition, the second PH electrode 330 may be not only easy to be installed, but also have a good sealing performance.

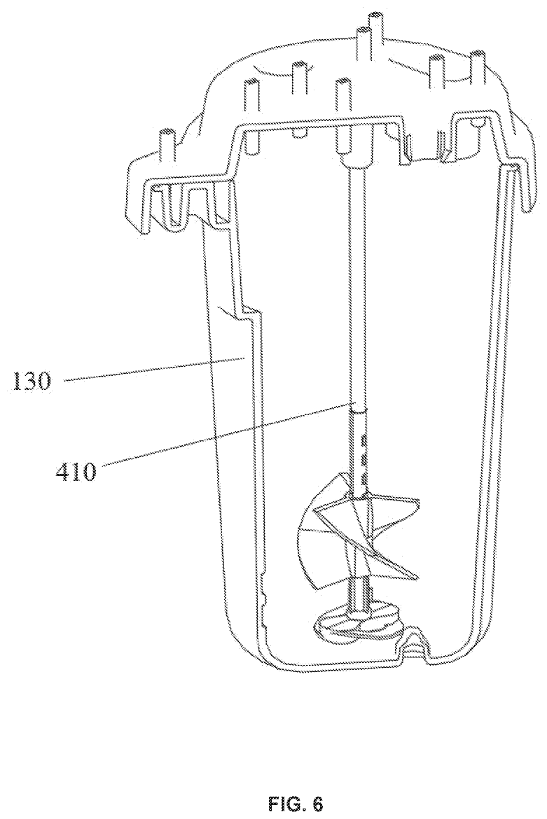

FIG. 6 is a schematic diagram illustrating an exemplary structure of an agitating device according to some embodiments of the present disclosure. As shown in FIG. 6, in some embodiments, the biological reaction system 100 may further include an agitating device.

In some embodiments, the agitating device may include a central rotating shaft 410 and an agitating component. The central rotating shaft 410 may be rotatably arranged within the accommodating chamber 130, and the agitating component may include a plurality of agitating paddle assemblies.

The central rotating shaft 410 refers to a shaft used to bear a bending moment and a torque during the rotating work. In some embodiments, the central rotating shaft 410 may be usually in a shape of rod, and a cross-section of the central rotating shaft 410 may be of any shape. For example, the cross-section of the central rotating shaft 410 may be in a shape of circle, triangle, quadrilateral, polygon, or the like. In some embodiments, other components (e.g., blades) may be installed on the central rotating shaft 410 to achieve corresponding functions (e.g., an agitating function).

The agitating component may be configured to mix gases, liquids, and even suspended particles in solution evenly. In some embodiments, the agitating component may include the plurality of agitating paddle assemblies. The agitating paddle assembly may be an agitating paddle. In some embodiments, at least two agitating paddle assemblies may be spliced along an axis direction of the central rotating shaft 410 to form a spiral paddle.

In some embodiments, since an outer diameter of a helical ribbon is equal to a pitch of the spiral paddle, the spiral paddle requires less rotational speed compared with an ordinary paddle (e.g., the ordinary paddle may be a pitched-blade turbine), when microcarriers or cell clusters are suspended. The required rotational speed may be small, the generated shear force may be small, and the damage to the cells may be also small, which is beneficial for cultivating cells.

In some embodiments, the agitating paddle assembly may include an installation portion and a paddle portion arranged on the installation portion. In some embodiments, the installation portion may be connected with the central rotating shaft 410. In some embodiments, the installation portion may be in a circular installation part, and the circular installation part may be sleeved on the central rotating shaft 410.

In some embodiments, the installation portion may facilitate easy disassemble and assemble of the agitating paddle assembly and the central rotating shaft 410.

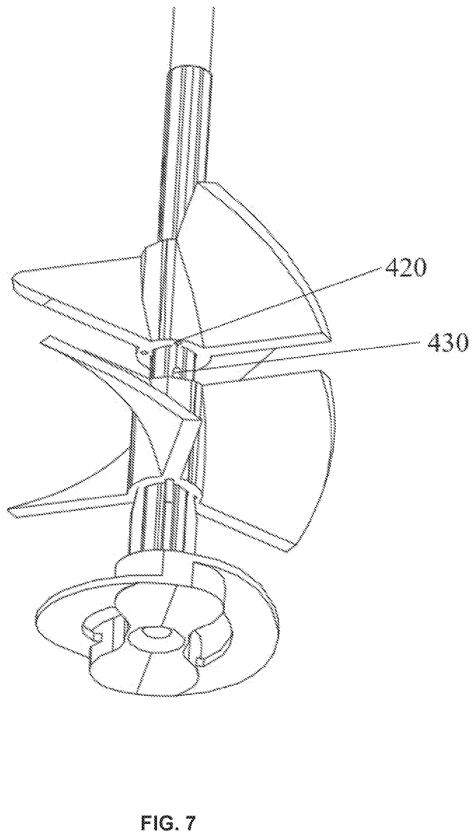

FIG. 7 is a schematic splicing diagram illustrating an agitating paddle assembly according to some embodiments of the present disclosure. As shown in FIG. 7, in some embodiments, for any two adjacent agitating paddle assemblies, a connecting groove 420 may be provided on the installation portion of one of the agitating paddle assemblies, and a connecting protrusion 430 may be provided on the installation portion of another agitating paddle assembly. The connecting protrusion 430 on the installation portion of the another agitating paddle assembly may be inserted into the connecting groove 420 on the installation portion of the agitating paddle assembly. For example, the connecting protrusion 430 and the connecting groove 420 may be connected through the snap-fit. In some embodiments, a single agitating paddle assembly may be a spiral paddle, and a size of the spiral paddle may be relatively small for easy injection molding manufacturing. In some embodiments, the plurality of agitating paddle assemblies may form a spiral paddle with a greater length along an extended direction of the central rotating shaft 410 after the plurality of agitating paddle assemblies are spliced through the connecting grooves and the connecting protrusions.

In some embodiments, the spiral paddle may be accurately spliced based on the connecting groove 420 and the connecting protrusion 430, thereby facilitating manufacturing and installation.

In some embodiments, the installation portion may be fixedly connected with the central rotating shaft 410. In some embodiments, the installation portion may be detachably connected with the central rotating shaft 410. For example, the detachable connection may be a snap-fit connection, a threaded connection, or the like. In some embodiments, the installation portion may be provided with a first fixing member, the central shaft 410 may be provided with a second fixing member, and the first fixing member and the second fixing member are connected through a snap-fit. In some embodiments, the first fixing member may be a positioning protrusion or a positioning groove, and the second fixing member may be a positioning groove or a positioning protrusion. It should be understood that the first fixing member may match the second fixing member. For example, when the first fixing member is a protrusion, the second fixing member may be a groove, and the protrusion may be flexibly stuck into the groove, so that the installation portion may be relatively fixed with the central rotating shaft 410.

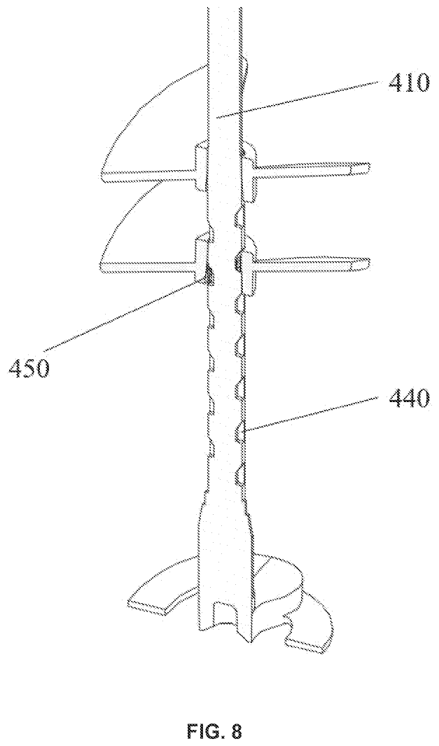

FIG. 8 is a schematic diagram illustrating an installation of an agitating paddle assembly according to some embodiments of the present disclosure. As shown in FIG. 8, in some embodiments, an inner wall of the installation portion may be provided with a positioning protrusion 450, and an outer wall of the central rotating shaft 410 may be provided with a positioning groove 440. The positioning protrusion 450 may be stuck into the positioning groove 440. In some embodiments, the inner wall of the installation portion may be provided with the positioning groove, the outer wall of the central rotating shaft 410 may be provided with the positioning protrusion, and the positioning protrusion may be stuck into the positioning groove.

In some embodiments, as shown in FIG. 8, an upper side and a lower side of the positioning protrusion 450 along the axis direction of the central rotating shaft 410 may be inclined surfaces, and a cross-sectional area of the positioning protrusion 450 along a height direction perpendicular to the positioning protrusion 450 may be gradually decreased from a bottom of the positioning protrusion 450 to a top of the positioning protrusion 450. In some embodiments, as shown in FIG. 8, the upper side and the lower side of the positioning groove 440 along the axis direction of the central rotating shaft 410 may be inclined surfaces, and a cross-sectional area of the positioning groove 440 along a depth direction perpendicular to the positioning groove 440 may be gradually decreased from the top of the positioning groove 440 to the bottom of the positioning groove 440. In some embodiments, the height direction of the positioning protrusion 450 may be parallel to the depth direction of the positioning groove 440, and the height direction of the positioning protrusion 450 may be perpendicular to the axis direction of the central rotating shaft 410.

In some embodiments, the arrangement of the structure of the positioning protrusion and positioning groove may make the positioning protrusion be stuck into the positioning groove smoothly along a direction of the positioning protrusion entering the positioning groove, thereby facilitating installation and disassembly.

In some embodiments, the inner wall of the installation portion may be provided with a plurality of positioning protrusions along a circumferential direction of the installation portion, and the outer wall of the central rotating shaft 410 may be provided with a plurality of positioning grooves along the circumferential direction of the central rotating shaft 410.

In some embodiments, the inner wall of the installation portion be provided with a plurality of positioning grooves along the circumferential direction of the installation portion, and the outer wall of the central rotating shaft 410 may be provided with a plurality of positioning protrusions along the circumferential direction of the central rotating shaft 410.

In some embodiments, the plurality of positioning grooves arranged on the outer wall of the central rotating shaft 410 may form a set of positioning grooves, and the outer wall of the central rotating shaft 410 may be provided with a plurality sets of positioning grooves along the axis direction of the central rotating shaft 410.

In some embodiments, the plurality of positioning protrusions arranged on the outer wall of the central rotating shaft 410 may form a set of positioning protrusions, and the outer wall of the central rotating shaft 410 may be provided with a plurality sets of positioning protrusions along the axis direction of the central rotating shaft 410.

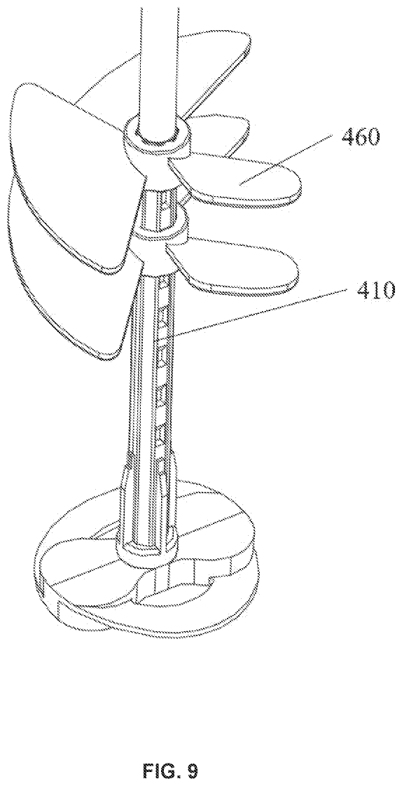

FIG. 9 is a schematic diagram illustrating another exemplary structure of an agitating device according to some embodiments of the present disclosure. As shown in FIG. 9, in some embodiments, the agitating component may also include a pitched-blade turbine 460, and the pitched-blade turbine 460 may be detachably connected with the central rotating shaft 410. In some embodiments, an installation manner of the pitched-blade turbine 460 on the central rotating shaft 410 may be similar to the spiral paddle illustrated above.

In some embodiments, the pitched-blade turbine may be used combined with the spiral paddle to improve an agitating effect.

In some embodiments, the biological reaction system 100 may further include a driving component, and the driving component may be configured to drive the agitating device to rotate.

In some embodiments, the central rotating shaft 410 may be rotatably arranged at the bottom of the reaction vessel 110.

In some embodiments, a structure used to support the agitating device may be provided at the bottom of the reaction vessel 110. In some embodiments, the bottom of the central rotating shaft 410 may be provided with a groove, and the bottom of the reaction vessel 110 may be provided with a column object. The coordination of the column object with the groove may prevent the central rotating shaft 410 from contacting the bottom of the accommodating chamber 130, and avoiding a generation of debris and impurities that affect the purity of the biological mixture through a friction between the central rotating shaft 410 and the bottom of the accommodating chamber 130 during rotation. In some embodiments, a wear-resistant member may be provided between the groove of the central rotating shaft 410 and the column object. For example, the wear-resistant member in a shape of hat may be sleeved on the column object. As another example, the wear-resistant member in a shape of ring may be sleeved on the column object. In some embodiments, the wear-resistant member may be made of flexible materials. For example, the wear-resistant member may be made of polypropylene, or the like. In some embodiments, the wear-resistant member may avoid a hard friction caused by a relative rotation between the column object and the groove of the central rotating shaft 410, thereby reducing the possibility of generating debris and impurities.

In some embodiments, the driving component may include a driving member, a first magnet, and a second magnet. The driving member may be connected with the first magnet to drive the first magnet to rotate along the axis direction of the central rotating shaft 410, and the second magnet may be connected with the central rotating shaft 410. The second magnet may drive the central rotating shaft 410 to rotate under a magnetic force of the first magnet.

In some embodiments, the driving component may include a motor or another driving device.

In some embodiments, the driving component may drive an operation of the agitating device through magnetic suction, which may not consider a dynamic sealing problem between the agitating device and the accommodating chamber 130, thereby reducing the leakage and pollution of the biological mixture.

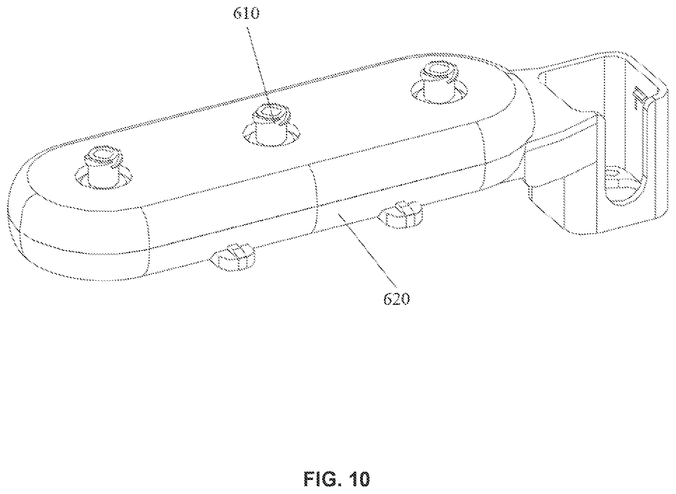

FIG. 10 is a schematic diagram illustrating an exemplary structure of a plurality of gas filters 610 according to some embodiments of the present disclosure. As shown in FIG. 10, in some embodiments, the biological reaction system 100 may include a plurality of gas filters 610, the plurality of gas filters 610 may be in communication with the accommodating chamber 130, and positions of the plurality of gas filters 610 may be relatively fixed to each other.

A gas filter 610 of the plurality of gas filters 610 may be configured to filter gas, remove bacteria and particles in the gas, or the like. The gas filter 610 may avoid external pollution of the internal environment of biological reaction system 100. In some embodiments, the gas filter 610 may include various forms and structures. For example, the gas filter 610 may be a bag filter, a disc filter, or the like, and a suitable gas filter may be selected as needed.

In some embodiments, the gas filter 610 may be in communication with the accommodating chamber 130, and the filtered gas may enter the accommodating chamber 130 after unneeded microorganisms and particles in the gas are filtered, so that a suitable environmental growth condition may be maintained within the accommodating chamber 130 by. In some embodiments, the biological reaction system 100 may be connected with a plurality of pipelines, and the plurality of pipelines may include but not limit to a surface gas pipeline, a deep gas pipeline, or the like. The plurality of pipelines may be connected with various external devices respectively.

In some embodiments, the plurality of gas filters 610 may be relatively fixed to each other, which may avoid experimental failures caused by misconnection of the pipelines. Additionally, the plurality of pipelines may be connected and disassembled as a whole, thereby making the operation simple and improving efficiency.

In some embodiments, the biological reaction system 100 may further include an installation member 620. The installation member 620 may include a plurality of fixing slots, and the plurality of gas filters may be fixed in the plurality of fixing slots, respectively, based on an one-to-one correspondence. In some embodiments, the fixing slots may be configured to fix positions of the plurality of gas filters 610, respectively, thereby preventing the plurality of gas filters 610 from moving.

In some embodiments, a count of the gas filters 610 may be at least three. The biological reaction system 100 may include a first connecting pipeline, a second connecting pipeline, and a third connecting pipeline. The first connecting pipeline, the second connecting pipeline, and the third connecting pipeline may be connected with three gas filters 610, respectively. In some embodiments, an end of the first connecting pipeline may be connected with a first gas filter 610, and another end of the first connecting pipeline may be in communication with a top of the accommodating chamber 130 to send in or send out the gas to the top of the accommodating chamber 130. An end of the second connecting pipeline may be connected with a second gas filter 610, and another end of the second connecting pipeline may be in communication with a bottom of the accommodating chamber 130 to send in or send out the gas to the bottom of the accommodating chamber 130. An end of the third connecting pipeline may be connected with a third gas filter 610, and another end of the third connecting pipeline may be in communication with a sampling port for sampling purposes. The sampling port may be in communication with the accommodating chamber 130.

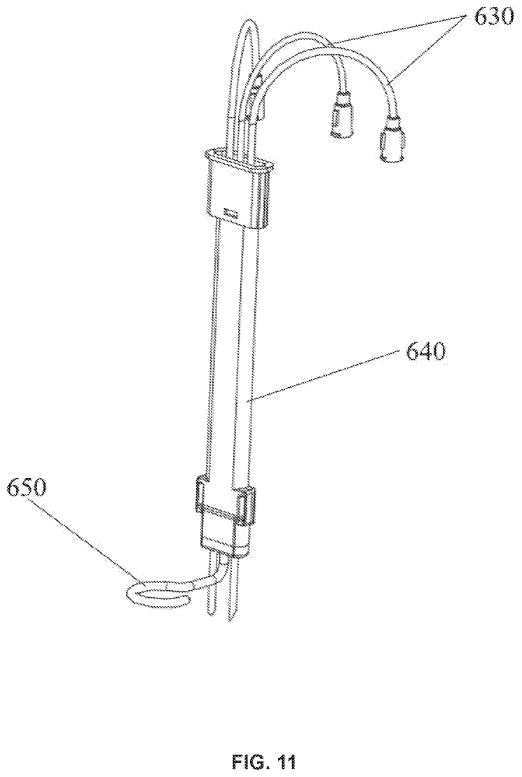

FIG. 11 is a schematic diagram illustrating an exemplary structure of a plurality of tracheas 630 and a trachea coupling member 640 according to some embodiments of the present disclosure. As shown in FIG. 11, in some embodiments, the biological reaction system 100 may further include a plurality of tracheas 630 and a trachea coupling member 640. The trachea coupling member 640 may be located within the accommodating chamber 130, and the plurality of tracheas 630 may be inserted into and penetrated through the trachea coupling member 640. In some embodiments, an end of each of the plurality of tracheas 630 may be located within the accommodating chamber 130. In some embodiments, an end of each of the plurality of tracheas 630 may be located at a bottom of the accommodating chamber 130, and another end of each of the plurality of tracheas 630 may be provided with a connector to connect a device as needed. In some embodiments, a count of the tracheas 630 may be at least four.

In some embodiments, the arrangement of the plurality of tracheas 630 in the trachea coupling member 640 may avoid a direct contact between the tracheas 630 and the mixture, and cells may be produced along gaps between the plurality of tracheas 630, thereby reducing the probability of cell wall hanging and clustering.

In some embodiments, one of the plurality of tracheas 630 may be a bottom communicating trachea, and the bottom communicating trachea may be configured to blow the gas to the bottom of the mixture. In some embodiments, an end of the bottom communicating trachea may be located at the bottom of the accommodating chamber 130. The end of the bottom communicating trachea located at the bottom of the accommodating chamber 130 may include a ring segment 650. The ring segment 650 facing a direction of the top of the accommodating chamber 130 may be provided with a plurality of openings, thereby achieving effective gas dissolution. In some embodiments, a diameter of the opening may be within a range of 0.2-1 mm to avoid excessive bubbles and bubble bursting.

In some embodiments, another end of the bottom communicating trachea may be in communication with the second connecting pipe to blow the gas into the accommodating chamber 130 through the second connecting pipe using the bottom communicating trachea.

In some embodiments, the biological reaction system 100 may also include a tail gas processing device, and the tail gas processing device may process the gas (e.g., tail gas) generated during cell growth and metabolism processes. In some embodiments, the tail gas processing device may include a tail gas processing filter 660 and a tail gas pipeline 670. An end of the tail gas pipeline 670 may be connected with the tail gas processing device, and another end of the tail gas pipeline may be configured to receive tail gas discharged from the accommodating chamber 130. In some embodiments, a plurality of tail gas pipelines 670 may be provided. In some embodiments, the tail gas pipeline 670 may include a plurality of pipeline openings, and diameters of the openings may be different. For example, one pipeline opening of the tail gas pipeline(s) 670 may receive the tail gas discharged from the accommodating chamber 130, and the other two or more pipeline openings of the tail gas pipeline(s) 670 may be connected with two or more tail gas processing filters 660, respectively.

In some embodiments, the tail gas processing device may balance the gas in the biological reaction system 100, and the generated harmful gas may be filtered through the tail gas processing filter 660. At the same time, the external pollution to the inside of the accommodating chamber 130 may also be avoided by connecting the tail gas processing filter 660.

In some embodiments, the tail gas processing device may further include a tail gas outflow channel, and the tail gas outflow channel may be in communication with the tail gas pipeline 670.

In some embodiments, the tail gas outflow channel may be arranged in various shapes, which may make the channel as long as possible with a small overall occupied space. When the tail gas passes through the tail gas outflow channel, a stay time may be relatively long, thus avoiding the blockage of the tail gas processing filter caused by water vapor in the tail gas based on relatively sufficient condensation.

In some embodiments, the tail gas processing device may further include a condenser, and the condenser may be configured to condense the tail gas passing through the tail gas processing device to avoid system failure caused by a high humidity of the tail gas blocking the filter. In some embodiments, the condenser may include a cooling element, and the cooling element may contact with a portion of the tail gas outflow channel. In some embodiments, the cooling element may contact with a channel wall of the tail gas outflow channel to enhance a condensation effect.

In some embodiments, the tail gas processing device may further include a heating member. The heating member may be arranged at an outer side of the tail gas processing filter 660, and the heating member may partially or completely wrap around the tail gas processing filter 660. The heating member may be configured to heat the tail gas processing filter 660, thereby causing liquid in the tail gas processing filter 660 to evaporate to solve the problem of filter blockage.

In some embodiments, the controller may determine an actual temperature value of a current mixture based on one or more parameters of a current measured temperature value, a depth of a groove, quantity of the mixture, and a thermal conductivity of the mixture, thereby eliminating possible measurement errors in an external measurement of the temperature sensor from the accommodating chamber 130, and obtaining a more realistic temperature. The measured temperature value refers to a temperature value obtained by the temperature sensor 120. The depth of the groove reflects an installation position of the temperature sensor. Generally, a suitable groove position may enable the temperature sensor to sense a temperature closer to the actual temperature of the mixture. The depth of the groove may be preset. That is, the biological reaction system may include the groove with a fixed depth, or the depth of the groove may also be measured. The quantity of the mixture refers to a volume of the mixture, a mass of the mixture, or the like, and the quantity of the mixture may be obtained by weighing or volumetric measurement, or the like. In some embodiments, the reaction vessel 110 may include a measurement scale used to measure the volume of the mixture. The thermal conductivity of the mixture refers to a thermal conductivity of the mixture in the reaction vessel 110. In some embodiments, the thermal conductivity may be equivalent to a thermal conductivity of a cell culture medium. A thermal conductivity system of the mixture may be measured using a thermal conductivity measuring instrument.

In some embodiments, the controller may determine the actual temperature value of the mixture based on one or more parameters of the measured temperature value, the depth of the groove, the quantity of the mixture, and the thermal conductivity of the mixture by using a temperature model. The temperature model may be a machine learning model. For example, the temperature model may be a neural network (NN), or the like. In some embodiments, an input of the temperature model may include the measured temperature value, the depth of the groove, the quantity of the mixture, and the thermal conductivity of the mixture, and an output of the temperature model may include the actual temperature value.

In some embodiments, the temperature model may be trained through a large count of first training samples with a first label. For example, the plurality of training samples with a training label may be input into the temperature model. A loss function may be constructed based on the training label and a prediction result of a preliminary temperature model. The preliminary temperature model may be iteratively updated based on the loss function, and training of the temperature model may be completed when the loss function of the preliminary temperature model satisfies a preset condition. The preset condition may be that the loss function converges, a count of iterations reaches a threshold, or the like.

The first training sample may include a sample measured temperature value, a sample depth of the groove, a sample quantity of the mixture, and a sample thermal conductivity of the mixture. The first label may be a sample actual temperature value. The first label may be manually labeled. The first training sample may be obtained through experiments. In some embodiments, the cell expansion experiment may be performed under a condition of the first training sample, a thermometer may be penetrated into the mixture multiple times to measure the temperature value, and a mean value may be designated as a first label corresponding to the first training sample.

In some embodiments, the controller may further control the heating mechanism 200 based on the actual temperature value output by the temperature model. In some embodiments, the controller may input the actual temperature value output by the temperature model, current heating temperature distribution information of the heating mechanism 200, a target temperature, the quantity of the mixture, and the thermal conductivity of the mixture into the heating model, and the heating model may output heating data of the heating mechanism 200 in a preset future period. The heating data may include a heating temperature and a heating time. The heating model may be a machine learning model. For example, the heating model may be a recurrent neural network (RNN) model.

In some embodiments, the heating mechanism 200 may include a heating assembly, and the heating assembly used for heating may be tightly adhered with the reaction vessel 110. Due to a relatively large surface area of the heating assembly, temperature non-uniformity may exist, thus, temperature sensing devices may be set up at a plurality of point positions of the heating assembly. The point positions may be set as needed. The point positions may be evenly distributed or randomly distributed. In some embodiments, a plurality of temperatures may be collected through temperature sensing devices at the plurality point positions. The current heating temperature distribution information of the heating mechanism 200 may include a corresponding setting position of each point position of the plurality of point positions, a temperature value collected at each point position, or the like. The heating temperature of the heating mechanism in the preset future period refers to a heating temperature of the heating mechanism 200 in the preset future period, and the heating time of the heating mechanism in the preset future period refers to a continuous heating time of the heating mechanism 200 in the preset future period. The heating temperature may be 0. That is, the heating mechanism may be turned off in the preset future time period and heating is not required. The heating time may include a time when heating starts and a time when heating stops. The heating time may also include a duration of the heating. In some embodiments, the heating temperature of the heating mechanism 200 in the preset future period output by the heating model may be the heating temperatures of one or more of the plurality of point positions of the heating assembly.

In some embodiments, the controller may control the heating mechanism 200 by outputting the heating temperature and the heating time of the heating mechanism 200 in the preset future period based on the heating model. For example, the heating temperature may be controlled by adjusting a power of the heating mechanism 200, and the heating time may be controlled by turning on and turning off the heating mechanism 200. In some embodiments, the heating assembly of the heating mechanism 200 may be composed of a plurality of heating members. Each heating member may be provided with a temperature sensing device, and the controller may control a power of each heating member to control a heating temperature of each heating member, thereby achieving the control of the heating temperature of the heating mechanism 200.

In some embodiments, an agitating speed of the agitating device may be selected from a preset rotating speed set. The preset rotating speed set refers to a data set formed by rotating speeds with a good effect during a biological reaction process. For example, the preset rotating speed set may be located within a rotating speed range that the cell growth condition is relatively good. The preset rotating speed set may include a plurality of rotating speed values or only a rotating speed value. In some embodiments, the preset rotating speed set may be constructed based on historical data. Merely by way of example, the preset rotating speed set may include an agitating speed of the agitating device in the historical data when the cells grow well.

In some embodiments, the input of the heating model may also include the agitating speed of the agitating device. In some embodiments, one or more rotating speeds may be selected from the preset rotating speed set to generate a plurality sets of candidate agitation rotating speeds, and the plurality sets of candidate agitation rotating speeds may be input into the heating model, respectively.

In some embodiments, the plurality sets of candidate agitation rotating speeds may correspond to a plurality of heating data (i.e., the heating temperature and the heating time) of the heating mechanism 200 output in the preset future period, respectively. The controller may calculate heating powers corresponding to each of the plurality of heating data based on the plurality of heating data, and select an agitation speed corresponding to heating data with a lowest heating power as an optimal agitation rotating speed. The controller may control the agitating device to rotate at the optimal agitation rotating speed during the future period.

The temperature model may compensate for a difference between the measured temperature value and the actual temperature value, thereby obtaining an accurate temperature inside the biological reaction system, which is beneficial for subsequent precise regulation. Furthermore, the heating model may determine the accurate heating temperature and heating time of the heating mechanism 200 based on the actual value output by the temperature model. The heating mechanism 200 may be regulated based on the heating data output by the temperature model to achieve precise temperature control of the biological reaction system. The optimal agitation rotating speed may be determined based on the heating power corresponding to the heating data, thereby reducing energy consumption.

The basic concepts have been described. Obviously, for those skilled in the art, the detailed disclosure may be only an example and may not constitute a limitation to the present disclosure. Although not explicitly stated here, those skilled in the art may make various modifications, improvements, and amendments to the present disclosure. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of the specification are not necessarily all referring to the same embodiment. In addition, some features, structures, or features in the present disclosure of one or more embodiments may be appropriately combined.

Moreover, unless otherwise specified in the claims, the sequence of the processing elements and sequences of the present application, the use of digital letters, or other names are not used to define the order of the application flow and methods. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various assemblies described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure and aiding in the understanding of one or more of the various embodiments. However, this disclosure may not mean that the present disclosure object requires more features than the features mentioned in the claims. In fact, the features of the embodiments are less than all of the features of the individual embodiments disclosed above.

In some embodiments, the numbers expressing quantities, properties, and so forth, used to describe and claim certain embodiments of the application are to be understood as being modified in some instances by the term “about,” “approximate,” or “substantially.” Unless otherwise stated, “about,” “approximate,” or “substantially” may indicate a ±20% variation of the value it describes. Accordingly, in some embodiments, the numerical parameters set forth in the description and attached claims are approximations that may vary depending upon the desired properties sought to be obtained by a particular embodiment. In some embodiments, the numerical parameters should be construed in light of the number of reported significant digits and by applying ordinary rounding techniques. Although the numerical domains and parameters used in the present application are used to confirm the range of ranges, the settings of this type are as accurate in the feasible range in the feasible range in the specific embodiments.

Each patent, patent application, patent application publication, and other materials cited herein, such as articles, books, instructions, publications, documents, etc., are hereby incorporated by reference in the entirety. In addition to the application history documents that are inconsistent or conflicting with the contents of the present disclosure, the documents that may limit the widest range of the claim of the present disclosure (currently or later attached to this application) are excluded from the present disclosure. It should be noted that if the description, definition, and/or terms used in the appended application of the present disclosure is inconsistent or conflicting with the content described in the present disclosure, the use of the description, definition and/or terms of the present disclosure shall prevail.

At last, it should be understood that the embodiments described in the disclosure are used only to illustrate the principles of the embodiments of this application. Other modifications may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A biological reaction system, comprising a reaction vessel and a temperature sensor, wherein
the reaction vessel includes an accommodating chamber configured to accommodate a mixture of cells and liquids;
an outer side of a bottom of the reaction vessel is provided with a groove recessed in a direction towards the accommodating chamber;
the temperature sensor includes a probe,
the probe is within the groove;
the groove is provided with a first heat conduction member, and
the first heat conduction member is sleeved outside the probe.

2. The biological reaction system of claim 1, further including a second heat conduction member, wherein
at least part of the second heat conduction member is located between the probe and the first heat conduction member,
a portion of an inner surface of the second heat conduction member is adhered with the probe, and
a portion of an outer surface of the second heat conduction member is adhered with an inner surface of the first heat conduction member.

3. The biological reaction system of claim 1, wherein the groove is further provided with a flexible sealing member, the sealing member is abutted against the first heat conduction member, and the sealing member is in an interference fit with the groove.

4. The biological reaction system of claim 1, wherein a ratio of a depth of the groove to a diameter of the bottom of the reaction vessel is within a range from 1:30-1:2.5.

5. The biological reaction system of claim 1, further including an elastic compaction mechanism, wherein
the elastic compaction mechanism includes a fixing portion, a compaction-abutting portion, and an elastic member,
a position of the fixing portion is fixed,
the elastic member is abutted between the fixing portion and the compaction-abutting portion, and
the compaction-abutting portion is configured to abut and press the first conduction member under an elastic force of the elastic member.

6. The biological reaction system of claim 1, wherein an inner wall of the first conduction member is adapted to a shape of the probe.

7. The biological reaction system of claim 6, wherein the inner wall of the first conduction is provided with an insulating layer, and the insulating layer is adhered with the probe.

8. The biological reaction system of claim 1, further including a heating mechanism and a controller, wherein
the heating mechanism is adhered with at least part of an outer wall of the reaction vessel,
the heating mechanism and the temperature sensor are connected with the controller, and
the controller controls a heating temperature of the heating mechanism based on a temperature sensed by the temperature sensor.

9. The biological reaction system of claim 1, further including a dissolved oxygen electrode and a first PH electrode, wherein the dissolved oxygen electrode and the first PH electrode are arranged within the accommodating chamber of the reaction vessel and located on a sidewall of the reaction vessel.

10. The biological reaction system of claim 1, further including a second PH electrode, wherein
the reaction vessel includes an upper cover configured to seal the accommodating chamber,
the upper cover includes a detecting port,
the second PH electrode is inserted into the accommodating chamber through the detecting port, and
a sealing device is arranged between the second PH electrode and the upper cover of the reaction vessel.

11. The biological reaction system of claim 1, further including a driving component and an agitating device, wherein
the agitating device includes a central rotating shaft and a plurality of agitating paddle assemblies,
for each of the plurality of agitating paddle assemblies,
the agitating paddle assembly includes an installation portion and a paddle portion arranged on the installation portion,
the installation portion is provided with a first fixing member,
the central rotating shaft is provided with a second fixing member, and
the first fixing member and the second fixing member are connected through a snap-fit.

12. The biological reaction system of claim 11, wherein
for any two adjacent agitating paddle assemblies of the plurality of agitating paddle assemblies,
a first installation portion of one agitating paddle assembly is provided with a connecting groove,
a second installation portion of another agitating paddle assembly is provided with a connecting protrusion, and
the connecting protrusion and the connecting groove are connected through the snap-fit.

13. The biological reaction system of claim 11, wherein
the first fixing member is a first positioning protrusion and the second fixing member is a first positioning groove, or the first fixing member is a second positioning groove and the second fixing member is a second positioning protrusion;
an inner wall of the installation portion is provided with the first positioning protrusion and an outer wall of the central rotating shaft is provided with the first positioning groove, and the first positioning protrusion is stuck into the first positioning groove, or
the inner wall of the installation portion is provided with the second positioning groove and the outer wall of the central rotating shaft is provided with the second positioning protrusion, and the second positioning protrusion is stuck into the second positioning groove.

14. The biological reaction system of claim 1, further including a plurality of gas filters, wherein the plurality of gas filters are in communication with the accommodating chamber, and positions of the plurality of gas filters are relatively fixed to each other.

15. The biological reaction system of claim 14, further including an installation member, wherein the installation member includes a plurality of fixing slots, and the plurality of gas filters are correspondingly fixed in the plurality of fixing slots, respectively.

16. The biological reaction system of claim 1, further including a plurality of tracheas and a trachea coupling member, wherein
the trachea coupling member is located within the accommodating chamber,
the plurality of tracheas are inserted into and penetrated through the trachea coupling member, and
one end of each of the plurality of tracheas is located within the accommodating chamber.

17. The biological reaction system of claim 16, wherein
one of the plurality of tracheas is a bottom communicating trachea,
an end of the bottom communicating trachea is located at a bottom of the accommodating chamber of the reaction vessel,
the end of the bottom communicating trachea located at the bottom of the accommodating chamber of the reaction vessel includes a ring segment, and
the ring segment facing a direction of a top of the accommodating chamber is provided with a plurality of openings.

18. The biological reaction system of claim 1, further including a tail gas processing device, wherein
the tail gas processing device includes a tail gas processing filter and a tail gas pipeline,
an end of the tail gas pipeline is connected with the tail gas processing device, and
another end of the tail gas pipeline is configured to receive tail gas discharged from the accommodating chamber.

19. The biological reaction system of claim 18, wherein the tail gas processing device further includes a tail gas outflow channel, and the tail gas outflow channel is in communication with the tail gas pipeline.

20. The biological reaction system of claim 18, wherein the tail gas processing device further includes a condenser and a heating member, the condenser is configured to condense tail gas passing through the tail gas processing device, and the heating member is configured to heat the tail gas processing filter.
